(19) United States
(12) Patent Application Publication
(10) Pub. No.: US 2021/0291019 A1
(43) Pub. Date: Sep. 23, 2021

(54) SMART HOME EXERCISE PROGRAM BASED ON TAG MANAGEMENT SYSTEM

(71) Applicant: CHENGDU SHANGYI INFORMATION TECHNOLOGY CO., LTD., CHENGDU (CN)

(72) Inventors: Xi CHEN, CHENGDU (CN); Youlin CHEN, CHENGDU (CN); Quan XIE, CHENGDU (CN); Runwen QI, CHENGDU (CN); Fengming YANG, CHENGDU (CN); Zijun GE, CHENGDU (CN)

(73) Assignee: CHENGDU SHANGYI INFORMATION TECHNOLOGY CO.

(21) Appl. No.: 17/015,204

(22) Filed: Sep. 9, 2020

(30) Foreign Application Priority Data
Mar. 19, 2020 (CN) 202010196300.7

(51) Int. Cl. A63B 24/00; G08B 21/18

(52) U.S. Cl. CPC A63B 24/0062; G08B 21/182; A63B 2220/803; A63B 2225/20; A63B 2220/62; A63B 2230/202; A63B 2230/04; A63B 2230/06

(57) ABSTRACT

The disclosure provides a smart home exercise program based on tag management, including a server, a management terminal, a HCP terminal and a patient terminal, wherein movements information is stored in the server, the management terminal is used to manage the movement information, tag the movement in multiple dimensions, and preset workouts for different health conditions as the system templates; the medical staff set a new workout or modify the system template, combine the workout with the principles of exercise prescription as a whole exercise program and transmitting the workout to the patient terminal through the HCP terminal; the patient execute the home exercise program follow the multimedia guide with remote monitoring by the patient terminal.

### CROSS-REFERENCE TO RELATED APPLICATIONS

This application claims priority to Chinese Patent Application No. 202010196300.7 with a filing date of Mar. 19, 2020. The content of the aforementioned applications, including any intervening amendments thereto, are incorporated, herein by reference.

### TECHNICAL FIELD

The disclosure relates to the technical field of tele-medicine, exercise-based rehabilitation, home exercise program (HEP), particularly to a personalized home exercise program based on tag management system.

### BACKGROUND OF THE PRESENT INVENTION

Exercise-based rehabilitation has emerged as a standard intervention for patients with kinds of health conditions. Specifically, exercise-based rehabilitation program is strong recommended for people with chronic diseases since it increases physical function, improves quality of life, and lowers hospital admission rates. Despite this, participation in exercise-based rehabilitation program remains low. Reported barriers to participation include transport difficulties, financial cost, embarrassment about participation, and program availability. Home exercise program has been proved an alternative approach that could alleviate some of these barriers. However, home-based exercise rehabilitation still has a lot of problems to be solved.

First, medical staffs need a tool to teach patients to do the specific movements without face-to-face therapy. Second, patients need an assistant to tell them whether they reach the requirement or not, specifically the exercise intensity that is directly related the safety and effectiveness of patients with chronic diseases. Actually, there are some home exercise programs based on videos and remote heart rate monitor to solve these problems, but nearly all these programs are fixed workouts to a group of patients for general purpose. However, patients with different health conditions need different exercise programs which are significantly related to the prognosis. Even patients with the same disease, different exercise program is required because of the discrepancy in progression of disease and exercise capacity. Therefore, a personalized exercise-based rehabilitation program with customized video course and remote monitor is in deed. Preferably, it can be ‘smart’ enough to push the customized exercise course automatically based on evidences for medical staffs to choose, as well as alarm in time according to the real-time feedback from patients.

### SUMMARY OF PRESENT INVENTION

To provide a better solution to exercise-based rehabilitation, the disclosure discloses a smart home exercise program based on tag management system. Thousands of movements can be combined into different workouts according to the various health conditions of different patients as well as the diverse goals of rehabilitation by adopting the system, thereby significantly improving the specialization and the variety of the exercise program on different clients.

The above objective of the disclosure is achieved through the following technical solution:

A smart home exercise program based on tag management, comprising a server, a management terminal, a health care provider (HCP) terminal and a patient terminal which are mutually communicated;

movements are stored in the server, and each movement has multidimensional tags; the server receives personalized exercise program from the HCP terminal and transmits the workout to the patient terminal;

the management terminal is used to manage the tags of each movements, add new movement and edit the existing movements; meanwhile the authorized staff can preset workouts through the management terminal for different health conditions and transmit the workouts to the server as the system templates;

the HCP terminal is used for setting a new workout or modifying the system template and transmitting the workout to the server.

the patient terminal is used for receiving workout and playing the multimedia workout to the client.

Preferably, the information of each movement comprises visual materials, specifically the videos, as well as audio and literal instructions including movement names, health effects and practice tips.

Preferably, the movement tags comprise applicable tags and inapplicable tags, the dimensions of applicable tags comprise type, intensity, pattern, body parts, instrument; the dimensions of inapplicable tags comprise diagnosis and/or pain.

Preferably, the workout has two different modes. One is fixed workout with specified movements that won't be changed unless the medical staff modify the workout when needed. Another one is smart workout with an editable tag formula that movements matching specified tags are randomly selected according to the tag formula. Movements can be automatically changed follow the formula, but cannot be replaced by the movements not matching the required tags.

Preferably, the workout includes the following phases: warm-up, functional training, cardio mix, cool-down and stretching; each phase comprises one or several groups of movements with the information on repetitions or time of each movement, circuit times of each group, as well as the interval time between movements.

Preferably, the exercise program comprises the principles of exercise prescription and the workout. Specifically, principles of exercise prescription includes frequency, intensity (target heart rate range), duration (exercise time per session), volume (exercise time per week), progression. The patient terminal will alarm when real-time heart rate detected by heart rate monitor is beyond the upper limitation during the whole exercise session, but it will alarm only in cardio mix phrase when heart rate doesn't reach the lower limitation.

Preferably, the HCP terminal and the patient terminal can be application on smart phone, tablet computer or PC terminal.

Preferably, the patient terminal can connect with intelligent hardware such as heart rate monitor, blood pressure monitor, glucometer, electrocardiogram (ECG), etc. The patient terminal demonstrates the physiological data detected by hardware and transmit them to the server that the medical staff can see the data on the HCP terminal.

Compared with the prior art, the disclosure has the following advantages:

1. The disclosure includes the server, the management terminal, the HCP terminal and the patient terminal. Movements are stored in the server like bricks. Authorized staffs manage the movements through the management terminal like classifying the bricks in different catalogs, add new movements, edit the information of existing movements, tag the movements in multiple dimensions and preset workout by specified movements or tag formula as the system preset template in the server. The medical staffs set workout for a correlated patient by selecting the movements according to combination of tags, or by choosing and modifying the system template through the HCP terminal, meanwhile the medical staffs set the principles of exercise prescription through the HCP terminal. The principles and the workout are combined as an integrated exercise program. Thus, different patients can complete their own exercise programs respectively through the patient terminals. Compared with the prior art, in the disclosure, we established a remote doctor-patient interactive exercise rehabilitation system based on tag management which makes the personalized home exercise program for different patients possible. By combining the specifically separated movements together like playing Legos, the personalization of video course can be maximized that is important to the efficacy and the safety for people with different health conditions. By choosing movements through tags or system templates, the efficiency of carrying out the home exercise program can be greatly improved.

2. The information of each movement comprises not only visual materials, specifically the videos, but also audio and literal instructions including movement names, health effects and practice tips, so as to help the patients comprehensively understanding of the movement and improve the accuracy of motor execution without face to face therapy.

3. The movement tags of the disclosure include the applicable tags and the inapplicable tags. The movements are selected according to the applicable tags and are excluded according to the inapplicable tags. When setting smart workout by tag formula, the workout can be more targeted to the main health problem by positive selecting, meanwhile it can avoid the movements which are contraindications in that health condition by negative selecting.

4. A single exercise session of the disclosure includes warm-up and function training unit, cardio mix training unit, cool-down and stretching unit. Different unit has different function, specifically in the requirement of exercise intensity and voice prompt. Thus, medical staff can set all kinds of exercise prescription according to the goal of rehabilitation.

5. The disclosure adopts the HCP terminal and the patient terminal, including the smart phone, the tablet computer or the PC terminal which has relevant control software, which can greatly reduce the difficulty of popularizing the system and facilitate the promotion.

6. The patient terminal of the disclosure can communicate with intelligent monitor for automatically collecting data that is uploaded and stored in the server. The intelligent monitors include heart rate monitor, blood pressure monitor, blood oxygen monitor, single-lead ECG or multi-leads ECG, so as to collect the patient' physiological data, which on the one hand improve the exercise safety by warn and/or stop the program automatically when detected the abnormal data, and on the other hand, make the remote optimization of the home exercise program by medical staff possible since the physiological data is synchronized in the HCP terminal through the server.

7. The workout of the disclosure includes two different modes, fixed workout with unchanged movements and smart workout with various movements that are randomly selected follow a settable rule with tags. The fixed workout is suitable for the patient who needs specific movements that cannot be replaced by other movements, and the smart workout is suitable for the patient who needs variety more than specificity. Owing to the different workout modes, the exercise program can adapt to different user requirements.

8. The patient terminal of the disclosure is also provided with the real-time alarm module. Prompt is given to the patients when in overload exercises, specifically the over intensity and/or overtime, thereby avoiding the injury of overload exercises on patients.

### FIGURE LEGENDS

FIG. 1 is a diagram of the structure of the disclosure;

FIG. 2 is a diagram of the workflow of the disclosure;

Reference numbers: 1, server; 2, management terminal; 3, HCP terminal; 4, patient terminal; 5, intelligent monitor

### DETAILED DESCRIPTION OF PREFERRED EMBODIMENTS

The disclosure will be further described in combination with figures and embodiments. Execution modes of the disclosure are not limited to the following embodiments.

### Embodiment 1

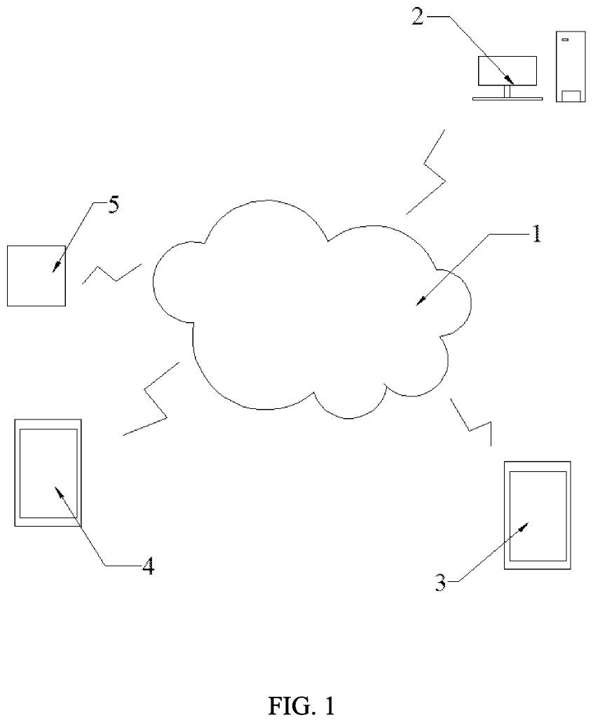

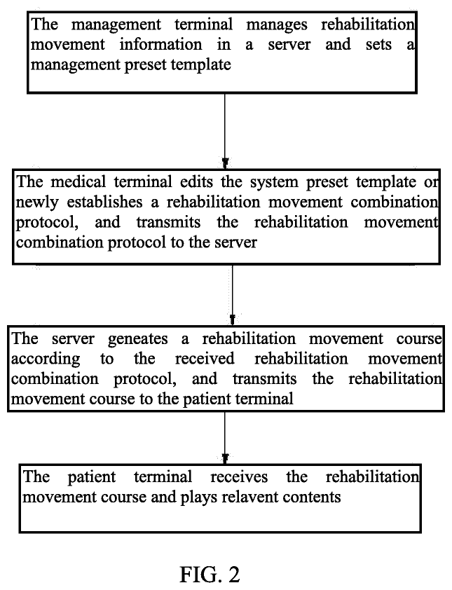

As a basic embodiment of the disclosure, this embodiment discloses a basic home exercise program based on tag management, as shown in FIG. 1 and FIG. 2, which includes a server 1, a management terminal 2, a HCP terminal 3 and a patient terminal 4, wherein the server 1 can use a cloud server or a self-built server, and the management terminal 2, the HCP terminal 3 and the patient terminal 4 can adopt a smart phone, a PC terminal or a tablet computer which is equipped with relevant softwares according to actual situations, and communication among the server 1, the management terminal 2, the HCP terminal 3 and the patient terminal 4 is realized through the Internet; a large number of separated movements is stored in the server 1, the above movements includes not only movement videos but also audio and literal instructions that are used for explaining movement essentials in detail, the authorized staff manage the movements information stored in the server 1 through the management terminal 2 (including addition, deletion and modification). At the same time, the management staff tag the same movements in multi-dimensions; the tags include applicable tags and inapplicable tags, wherein the evaluation dimensions of the applicable tags include types, intensity, pattern, body parts, instrument and the like; while the dimensions of the inapplicable tags include diagnosis, pain parts and other aspects;

When in use, the patient terminal 4 is bound with the unique HCP terminal 3, while the HCP terminal 3 can be simultaneously bound with multiple patient terminals 4; the medical staff select the wanted movements through the tags of each movements from movements data bank by the HCP terminal 3, put them in different units of a single exercise session, set repetition, groups and other parameters of the workout, combine the workout and the principles of exercise prescription as a program, then uploads the program to the server 1, the server 1 transmits the program to the patient terminal 4, and the patient execute the exercise program follow the multimedia instruction.

### Embodiment 2

As a preferred embodiment of the disclosure, this embodiment discloses a smart home exercise program based on tag management, as shown in FIG. 1 and FIG. 2, including a server 1, a management terminal 2, a HCP terminal 3 and a patient terminal 4; separated movements is stored in the server 1, the movements information includes not only movement videos but also audio and literal instructions that are used for explaining movement essentials in detail, the authorized staff manage the movements information stored in the server 1 through the management terminal 2 (including addition, deletion and modification). At the same time, the authorized staff tag the same movements in multi-dimensions; the tags include applicable tags and inapplicable tags, wherein the evaluation dimensions of the applicable tags include types, intensity, pattern, body parts, instrument and the like; while the dimensions of the inapplicable tags include diagnosis, pain parts and other aspects; the authorized staff can preset fix workouts with specified movements and/or smart workouts based on tag formulas through the management terminal 2 for different health conditions and transmit the workouts to the server 1 as the system templates.

When in use, the patient terminal 4 is bound with the unique HCP terminal 3, while the HCP terminal 3 can be simultaneously bound with multiple patient terminals 4; the medical staffs set workout for a correlated patient by selecting the movements according to combination of tags for the patient who needs specific movements, or by choosing and modifying the system template for the patient who needs variety more than specificity through the HCP terminal 3, then combine the workout and the principles of exercise prescription as a program, uploads the program to the server 1; the server 1 transmits the program to the patient terminal 4, and the patient execute the exercise program follow the multimedia instruction while automatically upload the real-time physiological data to the server 1 by intelligent monitor 5, such as the heart rate monitor, the blood pressure monitor, the single-lead ECG, etc.; the medical staff check the data and optimize the exercise program through the HCP terminal 3.

Further, the patient terminal 4 has an alarm module, and the medical staff can set the upper limit values of patient's key physiological indexes such as heart rate and blood pressure while setting the workout. When the data detected by the above intelligent monitors 5 exceeds the upper limit value or below the lower limit, the alarm module can simultaneously give alarms to the patient terminal 4 and the HCP terminal 3 to prevent the medical emergency.

Compared with the prior art, the disclosure realizes the personalized and dynamically optimized home exercise program with remote monitoring and doctor-patient interaction based on tag management system. With the tag management system, the separated movements can be quickly selected and freely combined into flexible and diversified workout according to the health condition of the patient. It can significantly improve the efficiency of exercise rehabilitation with system templates. Additionally, the disclosure establishes a remote communication channel between the patient and the medical staff, which is convenient for the medical staff to continual optimize the home exercise program based on the real-time feedback data.

We claim:

1. A smart home exercise program based on tag management, comprising a server (1), a management terminal (2), a HCP terminal (3) and a patient terminal (4) which are mutually communicated;
movements are stored in the server (1), and each movement has multidimensional tags; the server receives personalized exercise program from the HCP (3) terminal and transmits the workout to the patient terminal (4);
the management terminal (2) is used to manage the tags of each movements, add new movement and edit the existing movements; meanwhile the authorized staff can preset workouts through the management terminal (2) for different health conditions and transmit the workouts to the server (1) as the system templates;
the HCP terminal (3) is used for setting a new workout or modifying the system template and transmitting the workout to the server (1);
the patient terminal (4) is used for receiving workout and performing multimedia playing to a client.

2. The smart home exercise program based on tag management system according to claim 1, wherein the information of each movement comprises visual materials, specifically the videos, as well as audio and literal instructions including movement names, health effects and practice tips.

3. The smart home exercise program based on tag management according to claim 1, wherein the movement tags comprise applicable tags and inapplicable tags, the dimensions of applicable tags comprise type, intensity, pattern, body parts, instrument; the dimensions of inapplicable tags comprise diagnosis and/or pain.

4. The smart home exercise program based on tag management according to claim 1, wherein the program comprises the principles of exercise prescription and the workout. Specifically, principles of exercise prescription includes frequency, intensity (target heart rate range), duration (exercise time per session), volume (exercise time per week), progression; the patient terminal will alarm when real-time heart rate detected by heart rate monitor is beyond the upper limitation during the whole exercise session, but it will alarm only in cardio mix phrase when heart rate doesn't reach the lower limitation. The workout includes the following phases: warm-up, functional training, cardio mix, cool-down and stretching; each phase comprises one or several groups of movements with the information on repetitions or time of each movement, circuit times of each group, as well as the interval time between movements.

5. The smart home exercise program based on tag management according to claim 1, wherein the HCP terminal (3) and the patient terminal (4) can be application on smart phone, tablet computer or PC terminal.

6. The smart home exercise program based on tag management according to claim 1, wherein the patient terminal (4) is connected with intelligent monitors (5) such as heart rate monitor, blood pressure monitor, glucometer, electrocardiogram (ECG), etc. The patient terminal (4) demonstrates the physiological data detected by intelligent monitors and transmit them to the server (1) that the medical staff can see the data on the HCP terminal (3).

7. The smart home exercise program based on tag management according to claim 1, wherein the system formula preset by authorized staff through the management terminal (2) has two different modes. One is fixed workout with specified movements that won't be changed unless the medical staff modify the workout when needed. Another one is smart workout with an editable tag formula that movements matching specified tags are randomly selected according to the tag formula. Movements can be automatically changed follow the formula, but cannot be replaced by the movements not matching the required tags.

8. The smart home exercise program based on tag management according to claim 1, wherein the patient terminal (4) is also provided with an alarm module.
